SEQUENCE LISTING (1 sequence)

SEQ ID NO: 1 - DNA, 1377 bp, synthetic construct
  source 1..1377: mol_type = other DNA
aaggggttgggccaccggcttcgggtgttaccgactttcgtgacgtgacgggcggtgtgtacaaggcccgggaacgtattcaccgcagcaatgctgatctgcgattactagcgactccgacttcatggggtcgagttgcagaccccatccgaactgagaccggcttttttgagattcgctccaccttgcggtatcgcagctcattgtaccggccattgtagcacgtgtcagcccaagacataaggggcatgatgacttgacgtcgtccccaccttcctccgagttgaccccggcggtctcccgtgagtccccaacctccgaagagttgctggcaacacggacaagggttgcgctcgttgcgggacttaacccaacatctcacgacacgagctgacgacagccatgcaccacctgtacaccgaccacaaggggggcaccatctctgatgctttccggtgtatgtcaagccttggtaaggttcttcgcgttgcgtcgaattaagccacatgctccgccgcttgtgcgggcccccgtcaattcctttgagttttagccttgcggccgtactccccaggcggggcacttaatgcgttagctgcggcacggacaacgtggaatgttgcccacacctagtgcccaccgtttacggcgtggactaccagggtatctaatcctgttcgctccccacgcttcgctcctcagcgtcagtatcggcccagagatccgccttcgccaccggtgttcctcctgatatctgcgcatttcaccgctacaccaggaattccgatctcccctaccgaactctagcctgcccgtatcgactgcagacccggggttaagccccgggctttcacaaccgacgcgacaagccgcctacgagctctttacgcccaataattccggacaacgcttgcgccctacgtattaccgcggctgctggcacgtagttagccggcgcttcttctgcaggtaccgtcactctcgcttcttccctgctgaaagaggtttacaacccgaaggccgtcatccctcacgcggcgtcgctgcatcaggcttgcgcccattgtgcaatattccccactgctgcctcccgtaggagtctgggccgtgtctcagtcccagtgtggccggtcgccctctcaggccggctacccgtcgtcgccttggtgagccattacctcaccaacaagctgataggccgcgggctcatcctgcaccgccggagctttcgacccgccgagatgcctcggcaggtcagtatccggtattagaccccgtttccagggcttgtcccagagtgcagggcagattgcccacgtgttactcacccgttcgccactaatccccacccgaaggcggttcatcgttcgacttgca

(19) United States
(12) Patent Application Publication
(10) Pub. No.: US 2024/0032545 A1
(43) Pub. Date: Feb. 1, 2024

(54) STREPTOMYCES ARDESIACUS, MICROBIAL BIOLOGICAL CONTROL AGENT (MBCA) AND USE

(71) Applicant: Northwest A&F University, Xianyang (CN)

(72) Inventors: Lirong Han, Xianyang (CN); Zhichun Yuan, Xianyang (CN); Hao Wu, Xianyang (CN); Shanshan Xu, Xianyang (CN); Tingting Yu, Xianyang (CN)

(21) Appl. No.: 18/340,513

(22) Filed: Jun. 23, 2023

(30) Foreign Application Priority Data
Jul. 28, 2022 (CN) 202210898538.3

(51) Int. Cl. A01N 63/28; C12N 1/20; A01P 3/00; C05G 3/60

(52) U.S. Cl. CPC A01N 63/28; C12N 1/205; A01P 3/00; C05G 3/60; C12R 2001/465

(57) ABSTRACT

The present disclosure belongs to the technical field of microorganisms, and provides Streptomyces ardesiacus, a microbial biological control agent (MBCA) and use. The use specifically refers to prevention and treatment of various plant diseases. In the present disclosure, the Streptomyces ardesiacus has a serial number of HL-06, and has been deposited on Jun. 9, 2022, with a deposit number of CGMCC No. 25033. The HL-06 strain can degrade organic phosphorus and inorganic phosphorus, has a broad-spectrum antimicrobial activity, and can stimulate various defense enzymes of plants to improve disease resistance of the plants.

### TECHNICAL FIELD

The present disclosure belongs to the technical field of microorganisms, and in particular relates to Streptomyces ardesiacus, a microbial biological control agent (MBCA) and use.

### BACKGROUND

As a major producer of agricultural products, China has a small area of cultivated land, a high multi-cropping coefficient of crops, and a high intensity of cultivated land use. Naturally, China suffers from recurrent and frequent crop pests and diseases. At present, the control of plant diseases is still dominated by chemicals. However, excessive reliance on chemical pesticides has led to the destruction of ecological environment, the reduction of biodiversity, and the excessive pesticide residues. These factors bring adverse effects on environmental sustainability and human health. Due to environmental protection and no pollution, biological control is safe and harmless to people and environment. The biological control can effectively prevent and treat diseases, and has become a research hotspot in the control of plant diseases. Microbial inoculants have a simple production process. Most of the microbial inoculants can directly or indirectly improve soil, and have broad application prospects. Accordingly, there is a need to develop a microbial strain capable of suppressing the plant diseases.

### SUMMARY

To solve the above technical problems, the present disclosure provides Streptomyces ardesiacus, an MBCA and use.

A first objective of the present disclosure is to provide a Streptomyces ardesiacus HL-06, where the Streptomyces ardesiacus HL-06 has been deposited on Jun. 9, 2022, with a deposit number of CGMCC No. 25033.

A second objective of the present disclosure is to provide an MBCA, including the Streptomyces ardesiacus HL-06.

Preferably, the MBCA is a fermentation broth of the Streptomyces ardesiacus HL-06 or a microbial inoculant of the Streptomyces ardesiacus HL-06.

A third objective of the present disclosure is to provide a preparation method of the fermentation broth of the Streptomyces ardesiacus HL-06, including the following steps:
(1) conducting activation of a strain; and
(2) conducting seed preparation: preparing an obtained activated HL-06 strain into a spore suspension with a concentration of 10⁹ cfu/mL, inoculating the spore suspension in a sterilized seed culture solution according to an inoculum amount of 5% to 10% (w/w), with a volume in each bottle of 50 mL to 100 mL, and conducting shake cultivation at 28±2° C. for 3 d to 5 d to obtain a seed; where

the seed culture solution has a pH value of 7.2 to 7.6, and includes the following components in mass percentage: 0.1% to 2.0% of peptone, 1.0% to 3.0% of millet, 0.3% to 1.5% of glucose, 0.05% to 1% of NaCl, 0.05% to 2% of CaCO₃, and supplementing with water to 100%.

A fourth objective of the present disclosure is to provide a preparation method of the microbial inoculant of the Streptomyces ardesiacus HL-06, including the following steps:
(1) conducting activation of a strain; and
(2) conducting seed preparation: preparing an obtained activated HL-06 strain into a spore suspension with a concentration of 10⁹ cfu/mL, inoculating the spore suspension in a sterilized seed culture solution according to an inoculum amount of 5% to 10% (w/w), with a volume in each bottle of 50 mL to 100 mL, and conducting shake cultivation at 28±2° C. for 3 d to 5 d to obtain a seed; where
the seed culture solution has a pH value of 7.2 to 7.6, and includes the following components in mass percentage: 0.1% to 2.0% of peptone, 1.0% to 3.0% of millet, 0.3% to 1.5% of glucose, 0.05% to 1% of NaCl, 0.05% to 2% of CaCO₃, and supplementing with water to 100%; and
(3) conducting microbial inoculant preparation: inoculating the seed in a solid fermentation medium according to an inoculum amount of 5% to 30% under sterile conditions, conducting cultivation at 28±2° C. for 6 d, air-drying at 30° C., pulverizing, and adding an additive to obtain the microbial inoculant of the Streptomyces ardesiacus HL-06.

Preferably, the solid fermentation medium includes the following components in mass percentage based on a total percentage of 100%: 3% to 10% of corn flour, 10% to 30% of millet flour, 2% to 10% of potassium dihydrogen phosphate, and 35% to 60% of water.

Preferably, the additive includes diatomaceous earth equivalent to 2% to 10% of a mass fraction of the solid fermentation medium and sodium lauryl sulfate equivalent to 0.1% to 2% of the mass fraction of the solid fermentation medium. That is, the additive is a mixture of the diatomaceous earth and the sodium lauryl sulfate in the above dosages.

A fifth objective of the present disclosure is to provide use of the Streptomyces ardesiacus HL-06 or the MBCA in prevention and treatment of a plant disease.

Preferably, the plant disease is cucumber fusarium wilt, rice sheath blight, wheat sheath blight, apple Valsa canker, grape white rot, leaf blight on Schisandra, tomato gray mold, root rot of Schisandra, root rot of Scrophularia, or root rot of Panax notoginseng.

Preferably, the Streptomyces ardesiacus HL-06 or the MBCA are used for preparation of a drug, a fertilizer, or a medicinal fertilizer with an effect of controlling diseases. For example, a microbial solution of the Streptomyces ardesiacus HL-06 or the microbial inoculant of the Streptomyces ardesiacus HL-06 is mixed with nitrogen, phosphorus, and potassium fertilizers to obtain the medicinal fertilizer.

Preferably, the Streptomyces ardesiacus HL-06 or the MBCA or the medicinal fertilizer can be used to control root and leaf diseases of various crops by treatment methods including root irrigation, hole application, and spray treatment.

Compared with the prior art, the present disclosure has the following beneficial effects:

1. In the present disclosure, the Streptomyces ardesiacus has a serial number of HL-06. The strain can degrade organic phosphorus and inorganic phosphorus, has a broad-spectrum antimicrobial activity, and can stimulate various defense enzymes of plants to improve disease resistance of the plants.

2. In the present disclosure, a fermentation broth and a microbial inoculant of the HL-06 strain are prepared, and then subjected to experiments of antibacterial activity and disease control, respectively. The results show that both the fermentation broth and the microbial inoculant have desirable inhibitory and control effects on cucumber fusarium wilt, rice sheath blight, wheat sheath blight, apple Valsa canker, grape white rot, leaf blight on Schisandra, tomato gray mold, root rot of Schisandra, root rot of Scrophularia, or root rot of Panax notoginseng.

Deposit of Biological Material

The Streptomyces ardesiacus HL-06 has been deposited on Jun. 9, 2022 in the China General Microbiological Culture Collection Center (CGMCC) at NO. 1 West Beichen Road, Chaoyang District, Beijing 100101, with a deposit number of CGMCC No. 25033.

### BRIEF DESCRIPTION OF THE DRAWINGS

FIG. 1 shows a gene sequence of 16S rRNA of the Streptomyces ardesiacus HL-06; and

FIG. 2 shows a degradation effect of organic phosphorus (a) and inorganic phosphorus (b).

### DETAILED DESCRIPTION OF THE EMBODIMENTS

To enable a person skilled in the art to better understand technical solutions of the present disclosure, the present disclosure is further described below in detail with reference to the specific examples and accompanying drawings.

In the description of the present disclosure, unless otherwise specified, the reagents used are all commercially-available, and the methods used are conventional techniques in the art.

### Example 1: Strain Identification of Streptomyces ardesiacus HL-06

The Streptomyces ardesiacus HL-06 was inoculated on a plate of morphological observation mediums (including Czapek's medium, glucose aspartin medium, glycerol aspartin medium, inorganic salt starch medium, ISP-2 medium, oat flour medium, Gauze's synthetic medium No. 1, and Starkey's medium) by streaking, and subjected to inverted culture in a constant-temperature incubator at 28° C. On a 7th day, the colony morphology and soluble pigment changes of Streptomyces ardesiacus HL-06 on each medium were observed and recorded. The results were shown in Table 1.

TABLE 1
Culture characteristics of Streptomyces ardesiacus HL-06

| | | | Soluble |
|---|---|---|---|
| Medium | Aerial hyphae | Substrate hyphae | pigment |
| Czapek's medium | White | Lotus seed white | None |
| Glucose aspartin | Less, lychee white | Light milky yellow | None |
| medium | | | |
| Glycerol aspartin | Lychee white | Cinnamon light | None |
| medium | | brown | |
| Inorganic salt starch | White | Light clove brown | None |
| medium | | | |
| ISP-2 medium | White | Cork yellow | None |
| Oat flour medium | White to gray | Cork yellow | None |
| Gauze's synthetic | Lotus seed white | Yellowish-brown | None |
| medium No. 1 | | | |
| Starkey's medium | White | Cork yellow to | None |
| | | reddish brown | |

The Streptomyces ardesiacus HL-06 was inoculated on medium plates containing different carbon and nitrogen sources by a dot method, and the utilization of carbon and nitrogen sources was observed, and the results were observed after 7 d. The results were shown in Table 2.

Table 2 Physiological and biochemical characteristics of Streptomyces ardesiacus HL-06

| Feature | Results | Feature | Results | Feature | Results |
|---|---|---|---|---|---|
| Glucose | + | Trehalose | + | Sodium citrate | + |
| Mannose | − | Fructose | − | Sodium malate | + |
| Mannitol | + | Xylose | + | Sodium succinate | − |
| Lactose | + | Ribose | − | Sodium tartrate | − |
| Galactose | + | Synanthrin | + | Lysine | + |
| Sorbose | − | Starch | + | Threonine | + |
| Sorbitol | − | Arabinose | + | Alanine | + |
| Maltose | + | Cellobiose | + | Proline | + |
| Erythritol | − | Salicin | − | Nitrate reduction | + |
| Inositol | + | Dulcitol | − | Tyrosinase | + |
| Melezitose | + | Glycerol | + | Milk reaction | Peptoneization, |
| | | | | | non-coagulation |
| Rhamnose | + | Glycogen | + | | |
| Raffinose | + | Amygdalin | + | | |

Notes:
+, the test result was positive; −, the test result was negative.

As shown in Table 2, the Streptomyces ardesiacus HL-06 could not grow on the medium with mannose, sorbose, sorbitol, erythritol, fructose, ribose, salicin, dulcitol, sodium succinate, and sodium tartrate as a carbon source. The strain was able to utilize lysine, threonine, alanine, and proline as a nitrogen source. The strain was positive for nitrate reduction and tyrosinase reaction, and could peptonize milk without coagulation.

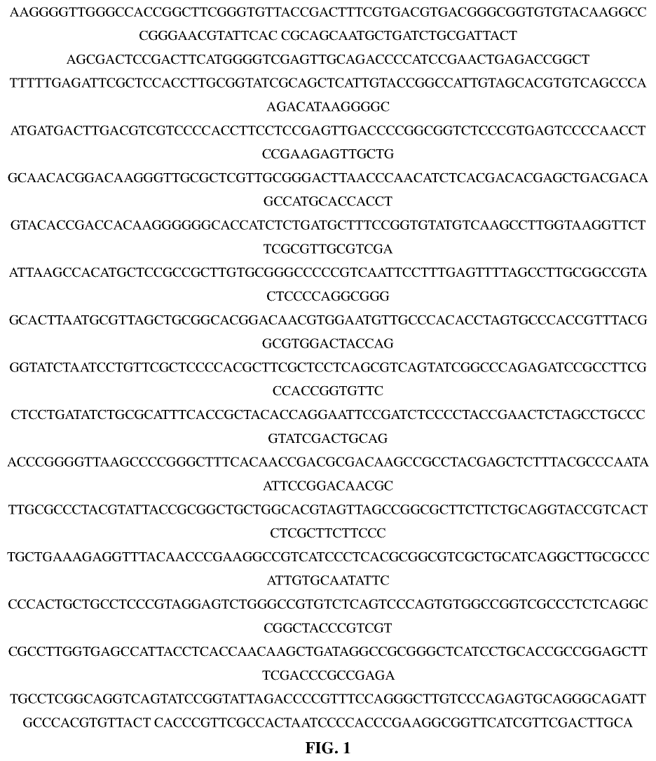

In this example, the Streptomyces ardesiacus HL-06 was isolated from actinomycetes in understory soil of the Qinling Mountains. According to the conservation of the following 16S rRNA gene sequence among microbial species, the Streptomyces ardesiacus HL-06 was identified. A genome was extracted from the Streptomyces ardesiacus HL-06, and 16S rRNA gene thereof was amplified by PCR and sequenced. The 16S rRNA gene sequence thereof was shown in SEQ ID NO. 1 and FIG. 1.

It is indicated that the strain belongs to Streptomyces ardesiacus of genus Streptomyces, and the strain was named Streptomyces ardesiacus HL-06 accordingly.

### Example 2: Determination of Phosphorus-Solubilizing Ability of Streptomyces ardesiacus HL-06

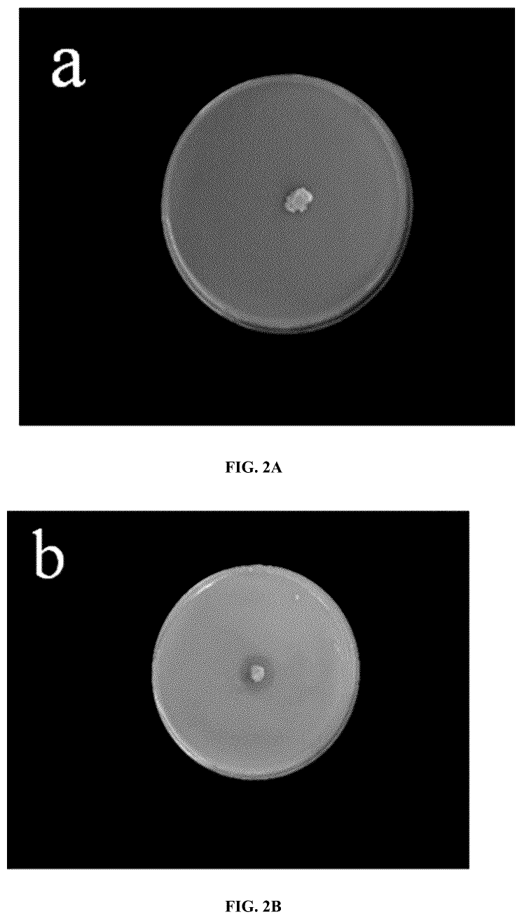

A phosphate-dissolving medium was prepared, including an inorganic phosphorus-dissolving medium and an organic phosphorus-dissolving medium. The bacterial cake (d=6.0 mm) of a biocontrol strain to be tested was inoculated in a center of the corresponding medium, and placed in a 28° C. incubator for 5 d. The ability of the strain to decompose inorganic phosphorus and organic phosphorus was detected. If there was a transparent circle around the strain, it meant that the strain showed the ability to decompose phosphorus. The results showed that the HL-06 produced transparent circles on the phosphate-dissolving medium (organic phosphorus and inorganic phosphorus), indicating that the strain could dissolve organic phosphorus and inorganic phosphorus. The determination results of the phosphorus-dissolving properties of the HL-06 strain were shown in FIG. 2.

The formulation of the inorganic phosphorus-dissolving medium included (in mass percentage): (NH₄)₂SO₄ 0.05%, NaCl 0.035%, KCl 0.035%, MgSO₄·7H₂O 0.03%, Ca₃(PO₄)₂ 0.50%, FeSO₄·7H₂O 0.003%, MnSO₄·H₂O 0.003%, glucose 1.0%, agar powder 2%, and supplementing with distilled water to 100%.

The formulation of the organic phosphorus-dissolving medium included (in mass percentage): (NH₄)₂SO₄ 0.05%, MgSO₄·7H₂O 0.03%, NaCl 0.03%, KCl 0.03%, CaCO₃ 0.35%, glucose 1.0%, MnSO₄·H₂O 0.003%, FeSO₄·7H₂O 0.003%, lecithin 0.02%, agar powder 2.0%, and supplementing with distilled water to 100%.

### Example 3: Determination of Antimicrobial Activity of Fermentation Broth of Streptomyces ardesiacus HL-06

1. A preparation method of a fermentation broth of the Streptomyces ardesiacus HL-06 included the following steps:
(1) conducting activation of a strain: the Streptomyces ardesiacus HL-06 was subjected to activation culture at 28° C. with Gauze's synthetic medium No. 1; and
(2) conducting seed preparation: an obtained activated HL-06 strain was prepared into a spore suspension with a concentration of 10⁹ cfu/mL, the spore suspension was inoculated in a sterilized seed culture solution according to an inoculum amount of 5.0% (w/w), with a volume in each bottle of 75 mL, and shake cultivation was conducted at 180 r/min and 28° C. for 3d to obtain a seed; where
the seed culture solution had a pH value of 7.2, and included the following components in mass percentage: 0.1% of peptone, 1.5% of millet, 1.0% of glucose, 0.5% of NaCl, 1.0% of CaCO₃, and supplementing with water to 100%.

2. An antagonistic activity of the fermentation broth of the Streptomyces ardesiacus HL-06 to 12 kinds of pathogenic bacteria for testing was determined by confrontation method. A PDA plate was prepared, a bacterial cake of the Streptomyces ardesiacus HL-06 was inoculated on the four sides of the plate at a distance of 30.0 mm from the center after the medium was solidified, and a bacterial cake (d=6.0 mm) for each of different pathogenic bacteria was inoculated in the center of the plate after 3 d. A plate only inoculated with the pathogenic bacteria to be tested was used as a control, and each treatment was repeated 3 times. Cultivation was conducted in an incubator at 25° C., the results were determined when the hyphae of the colonies on the control plate were about to grow to the edge of the petri dish, and an inhibition rate was calculated. The results were shown in Table 3. The Streptomyces ardesiacus HL-06 had a certain antifungal activity against the 12 kinds of plant pathogenic fungi to be tested. Moreover, the inhibition rates of the strains to Fusarium oxysporum f. sp. cucumebrium Owen, Fusa hum graminearum, and Alternaria tenuissima (Nees: Fr.) Wiltshire were all above 90%.

$$Mycelial growth inhibition rate ( \% ) = \frac{\begin{matrix}Control colony diameter - \\ Treated colony diameter\end{matrix}}{Control colony diameter} ⨯ 100$$

Table 3 Inhibitory effect of Streptomyces ardesiacus HL-06 on 12 kinds of phytopathogenic fungi in a dish

| | Colony diameter | Inhibition |
|---|---|---|
| | (with HL-06) | rate |
| Pathogens tested | (mm) | (%) |
| Panax notoginseng root rot pathogen | 23.18 ± 0.82 | 74.24 ± 1.24 |
| Valsa ceratosperma (Tode: Fr.) Maire | 27.44 ± 0.61 | 69.51 ± 0.93 |
| Glomerella ciningulaia (Stonem.) | 14.48 ± 1.13 | 83.91 ± 1.55 |
| Spauld. et Sch. | | |
| Rhizoctonia solani Kühn | 9.78 ± 0.85 | 89.13 ± 0.99 |
| Fusarium oxysporum f. sp. | 7.66 ± 1.01 | 91.49 ± 1.83 |
| cucumebrium Owen | | |
| Scrophularia root rot pathogen | 15.14 ± 0.94 | 83.18 ± 1.26 |
| Botrytis cinerea Pers. ex Fr. | 29.46 ± 1.25 | 67.27 ± 1.73 |
| Coniella diplodiella Petrak et Sydow | 23.46 ± 0.72 | 73.93 ± 0.51 |
| Fusarium graminearum | 8.66 ± 1.43 | 90.38 ± 1.62 |
| Rhizoctonia cerealis | 9.36 ± 1.53 | 89.60 ± 2.15 |
| Alternaria tenuissima (Nees: Fr.) | 8.04 ± 1.72 | 91.07 ± 2.51 |
| Wiltshire | | |
| Fusarium spp. | 9.26 ± 1.31 | 89.71 ± 1.92 |

The source of bacterial strains in Table 3: the following bacterial strains were all preserved in the laboratory of the inventor team of the present disclosure:
(1) Fusarium oxysporum: GU Li-sha; JIA Zheng-yan; ZHANG Xiao, et al. Inhibition of Camphor Tree Leaf Extract on Panax notoginseng Root Rot [J]. Journal of Yunnan Normal University (Natural Sciences Edition), 2020, 40(06): 12-16.
(2) Valsa mali Miyabe et Yamada: Qingguo Meng, Jing Shen, Juanhua Dong, et al. Control Effects of Two Biocontrol Bacteria on Apple Canker [J]. Modernizing Agriculture, 2019(02): 6-7.
(3) Glomerella ciningulaia (Stonem.) Spauld. et Sch.: Yu Zhanjing, Hou Xiaojie, Gao Qunying, et al. Inhibitory effect of 6 kinds of bacteria on apple glomerella leaf spot [J]. Jiangsu Agricultural Sciences, 2022, 50(03): 121-124.
(4) Rhizoctonia solani Kühn: Songhong Wei, Yating Zhang, Lingchun Kong, et al. Screening of biocontrol bacteria for rice sheath blight and field control effect [J]. Pesticides, 2022, 61(7).
(5) Fusarium oxysporum f sp. cucumebrium Owen: Jing A O, Yang L I, Xiaohui L I U, et al. Screening and Identification of Antagonistic Bacteria against Fusarium Wilt of Cucumber and Preliminary Study on Its Antifungal Effect [J]. JOURNAL OF YUNNAN AGRICULTURAL UNIVERSITY (Natural Science), 2022, 37(03): 429-434.
(6) Scrophularia root rot pathogen: Jieyi Xiao, Xiaoqiang Ren, Lingxue Chen.
Investigation and research on the occurrence period and regularity of Scrophularia root rot [J]. Modern Agricultural Science and Technology. 2014, (03).
(7) Botrytis cinerea Pers. ex Fr.: PAN Xiaomei; LI Zhaoyu; SHI Xiaoling, et al. Screening, Identification and Biocontrol Effects of Antagonistic Bacterial Strain XF and Preliminary Exploration of Biocontrol Factors [J]. Acta Agriculturae Boreali-occidentalis Sinica. 2019, 28(11) 1888-1895.
(8) Coniella diplodiella Petrak et Sydow: LI Baoyan; SHI Jie; TIAN Yuanyuan, et al. The sensitivity to imazalil and cross-resistance against several other fungicides in grapevine white rot pathogen Coniella diplodiella [J]. Journal of Plant Protection. 2021, 48(04): 774-780.
(9) Fusarium graminearum: ZHANG Zhen; QIU Haiping; CHAI Rongyao, et al. Identification of a Biocontrol Bacterium Strain against Fusarium graminearum and Its Preliminary Study on Biocontrol Mechanism [J]. Chinese Journal of Biological Control. 2022, 38(03): 673-680.
(10) Rhizoctonia cerealis: JU Yu-liang; SHEN Peng-fei; FENG Yan-juan, et al. Development and application of Rc-RPA-LFD for the rapid detection of Rhizoctonia cerealis [J]. Acta Phytopathologica Sinica. 2020, 50(05): 618-621.
(11) Alter-naria tenuissmia (Fr.) Wiltshire and Fusarium proliferatum: Wang Zhuang, Zhang Aihua, Lei Fengjie, etc. Biological Characteristics of the Pathogen of Schisandra chinensis Leaf Blight and Screening of Antagonistic Actinomycete Strain against the Pathogen [J]. Journal of Jilin Agricultural University. 2015, 37(05): 524-529.

### Example 4: Determination of Microbial Inoculant of Streptomyces ardesiacus HL-06 to Potted Medicinal Effect of Several Kinds of Plant Root Rots

1. A preparation method of a microbial inoculant of the Streptomyces ardesiacus HL-06 included the following steps:
(1) conducting activation of a strain: the Streptomyces ardesiacus HL-06 was subjected to activation culture at 28° C. with a Gauze's synthetic medium No. 1; and
(2) conducting seed preparation: an obtained activated HL-06 strain was prepared into a spore suspension with a concentration of 10⁹ cfu/mL, the spore suspension was inoculated in a sterilized seed culture solution according to an inoculum amount of 5.0% (w/w), with a volume in each bottle of 75 mL, and shake cultivation was conducted at 180 r/min and 28° C. for 3.0 d to obtain a seed; where
the seed culture solution had a pH value of 7.2, and included the following components in mass percentage: 0.1% of peptone, 1.5% of millet, 1.0% of glucose, 0.5% of NaCl, 1.0% of CaCO₃, and supplementing with water to 100%.
(3) Conducting microbial inoculant preparation: the seed was inoculated in a solid fermentation medium according to an inoculum amount of 10% under sterile conditions, cultivation was conducted at 28° C. for 6 d, air-dried at 30° C., pulverized, and an additive was added to obtain the microbial inoculant of the Streptomyces ardesiacus HL-06.

2. A control effect of potted plants was determined by root injury inoculation, and the test was started when the plant seedlings grew 4 to 6 true leaves. The preventive effect included: 10 mL of a 400-fold dilution of the HL-06 microbial inoculant was uniformly inoculated in the rhizosphere of plants, cultured at 25° C. for 24 h, and inoculated with 10⁷/mL spore suspensions of pathogenic bacteria of different plant root rot diseases. The treatment test included: 1×10⁷/mL spore suspension of the root rot pathogen was inoculated, and then 10 mL of the 400-fold dilution of the HL-06 microbial inoculant was inoculated after 24 h. A 1000-fold dilution of a 25% carbendazim wettable powder (WP) for root-irrigation was used as a positive control, and sterile water for root-irrigation was used as a negative control. The treated seedlings were cultured at 25° C. under light/dark=12 h/12 h, where 12 pots were treated in each group, repeated 3 times, and the disease incidence was investigated 30 d after inoculation. The results were shown in Table 4. The 400-fold dilution of the HL-06 microbial inoculant had a protective effect of 68.42%, 57.23%, 62.16%, and 73.68%, and a therapeutic effect of 51.63%, 47.37%, 21.05%, and 54.12% on cucumber fusarium wilt, Scrophularia root rot, Schisandra root rot, or Panax notoginseng root rot, respectively.

A formula for calculating the control effect was:

$$Disease index = \sum \frac{\begin{matrix}( Number of plants with disease grade ⨯ \\ representative grade )\end{matrix}}{\begin{matrix}Total number of plants ⨯ \\ maximum representative level value\end{matrix}} ⨯ 100$$

$$Control effect = \frac{\begin{matrix}Control disease index - \\ Treated disease index\end{matrix}}{Control disease index} ⨯ 100$$

TABLE 4
Control effects of HL-06 microbial inoculant on
root rot of several plants at seedling stage

| | Protective effect | | Therapeutic effect | |
|---|---|---|---|---|
| | | Control | | Control |
| | Disease | effect | Disease | effect |
| Treatment | index | (%) | index | (%) |
| Cucumber | 25.00 ± 1.67 | 68.42 ± 2.55 | 37.50 ± 2.11 | 51.63 ± 2.23 |
| fusarium wilt | | | | |
| Scrophularia | 33.33 ± 1.32 | 57.23 ± 1.76 | 41.67 ± 1.76 | 47.37 ± 2.77 |
| root rot | | | | |
| Schisandra | 29.17 ± 2.45 | 62.16 ± 2.32 | 62.50 ± 2.11 | 21.05 ± 1.93 |
| root rot | | | | |
| Panax | 20.83 ± 1.67 | 73.68 ± 1.46 | 37.50 ± 1.78 | 54.12 ± 1.32 |
| notoginseng | | | | |
| root rot | | | | |
| 25% | 29.17 ± 3.02 | 64.16 ± 2.34 | 33.33 ± 0.99 | 58.89 ± 1.23 |
| carbendazim | | | | |
| WP | | | | |
| Blank control | 79.17 ± 1.15 | — | 78.52 ± 0.85 | — |

### Example 5: Determination of Field Efficacy of the Microbial Inoculant of Streptomyces ardesiacus HL-06 (Namely the Microbial Inoculant of the Streptomyces ardesiacus HL-06 Prepared in Example 4) to Schisandra Leaf Blight

The field control effect of the Streptomyces ardesiacus HL-06 on the Schisandra leaf blight was determined, and the results were shown in Table 5. The 100-fold, 200-fold, and 400-fold dilutions of the HL-06 microbial inoculant had control effects of 70.00%, 63.24% and 51.54%, respectively. As the dilution factor increased, the control effect decreased.

TABLE 5
Field control effects of Streptomyces ardesiacus HL-
06 microbial inoculant on Schisandra leaf blight

| | Disease | Disease | |
|---|---|---|---|
| | index before | index after | Efficacy |
| Treatment | administration | administration | (%) |
| 100-fold dilution | 1.09 | 2.64 | 70.00 ± 1.35 |
| of HL-06 microbial | | | |
| inoculant | | | |
| 200-fold dilution | 1.16 | 2.82 | 65.90 ± 3.38 |
| of HL-06 microbial | | | |
| inoculant | | | |
| 400-fold dilution | 1.18 | 3.94 | 51.54 ± 3.23 |
| of HL-06 microbial | | | |
| inoculant | | | |
| 500-fold dilution | 1.19 | 2.45 | 69.61 ± 3.35 |
| of 25% | | | |
| carbendazim WP | | | |
| Clean water as | 1.13 | 10.84 | — |
| control | | | |

Note:
“—” meant no control effect.

It should be noted that when a numerical range is involved in the present disclosure, it shall be understood that the two end points of each numerical range and any value between the two end points can be selected. Since the steps and methods used are the same as those in the examples, the present disclosure describes the preferred example in order to prevent repetition. Although some preferred examples of the present disclosure have been described, persons skilled in the art can make changes and modifications to these examples once they learn the basic inventive concept. Therefore, the appended claims are intended to be interpreted as including the preferred examples and all alterations and modifications falling within the scope of the present disclosure.

Obviously, those skilled in the art can make various alterations and modifications to the present disclosure without departing from the spirit and scope of the present disclosure. The present disclosure is intended to cover these modifications and variations provided that they fall within the scope of protection defined by the following claims and their equivalent technologies.

What is claimed is:

1. A microbial biological control agent (MBCA), comprising a Streptomyces ardesiacus HL-06, wherein the Streptomyces ardesiacus HL-06 has been deposited on Jun. 9, 2022 in the China General Microbiological Culture Collection Center (CGMCC) at NO. 1 West Beichen Road, Chaoyang District, Beijing 100101, with a deposit number of CGMCC No. 25033.

2-10. (canceled)

11. The MBCA according to claim 1, wherein the MBCA is a fermentation broth of the Streptomyces ardesiacus HL-06 or a microbial inoculant of the Streptomyces ardesiacus HL-06.

12. The MBCA according to claim 11, wherein a preparation method of the fermentation broth of the Streptomyces ardesiacus HL-06 comprises the following steps:
(1) conducting activation of a strain; and
(2) conducting seed preparation: preparing an obtained activated HL-06 strain into a spore suspension with a concentration of 10⁹ cfu/mL, inoculating the spore suspension in a sterilized seed culture solution, and conducting shake cultivation at 28±2° C. for 3 d to 5 d to obtain a seed; wherein
the seed culture solution has a pH value of 7.2 to 7.6, and comprises the following components in mass percentage: 0.1% to 2.0% of peptone, 1.0% to 3.0% of millet, 0.3% to 1.5% of glucose, 0.05% to 1% of NaCl, 0.05% to 2% of CaCO₃, and supplementing with water to 100%.

13. A method of making a microbial inoculant for the MBCA of claim 1 comprising:
(1) conducting activation of the Streptomyces ardesiacus HL-06 strain; and
(2) conducting seed preparation by preparing the activated HL-06 strain into a spore suspension with a concentration of 10⁹ cfu/mL, inoculating the spore suspension in a sterilized seed culture solution, and conducting shake cultivation at 28±2° C. for 3 days to 5 days to obtain a seed; wherein
the sterilized seed culture solution has a pH value of 7.2 to 7.6, and comprises the following components in mass percentage: 0.1% to 2.0% of peptone, 1.0% to 3.0% of millet, 0.3% to 1.5% of glucose, 0.05% to 1% of NaCl, 0.05% to 2% of CaCO₃, and supplementing with water to 100%; and
(3) conducting microbial inoculant preparation by inoculating the seed in a solid fermentation medium under sterile conditions, conducting cultivation at 28±2° C. for 6 days to 8 days, air-drying at 30±2° C., pulverizing, and adding an additive to obtain the microbial inoculant of the Streptomyces ardesiacus HL-06.

14. The MBCA according to claim 13, wherein the solid fermentation medium comprises the following components in mass percentage based on a total percentage of 100%: 3% to 10% of corn flour, 10% to 30% of millet flour, 2% to 10% of potassium dihydrogen phosphate, and 35% to 60% of water.

15. The MBCA according to claim 13, wherein the additive comprises diatomaceous earth equivalent to 2% to 10% of a mass fraction of the solid fermentation medium and sodium lauryl sulfate equivalent to 0.1% to 2% of the mass fraction of the solid fermentation medium.

16. A method of using the MBCA according to claim 1 for the treatment of a plant disease, comprising: contacting a plant or a portion thereof, a plant's roots, or the soil near a plant with the MBCA of claim 1, wherein the plant disease is cucumber fusarium wilt, rice sheath blight, wheat sheath blight, apple Valsa canker, grape white rot, leaf blight on Schisandra, tomato gray mold, root rot of Schisandra, root rot of Scrophularia, or root rot of Panax notoginseng.

17. The method according to claim 16, wherein the MBCA is incorporated into a fertilizer, which is applied to a portion of the plant, the plant's roots, or the soil near the plant.
